(19) United States
(12) Patent Application Publication
(10) Pub. No.: US 2002/0076422 A1
(43) Pub. Date: Jun. 20, 2002

(54) CREAM CLEANSER WITH HIGH LEVELS OF EMOLLIENTS AND GLYCERIN

(76) Inventors: SNEHAL M. SHAH, ARTESIA, CA (US); GLENN A. NYSTRAND, LEBANON, NJ (US); MOLLY BETH CHARTIER, WEST HARTFORD, CT (US)

Correspondence Address: PHILIP S. JOHNSONN, ONE JOHNSON & JOHNSON PLAZA, NEW BRUNSWICK, NJ, 08933-7003, US

(21) Appl. No.: 09/417,552

(22) Filed: Oct. 13, 1999

Related U.S. Application Data
(60) Provisional application No. 60/104,106, filed on Oct. 13, 1998.

(51) Int. Cl. A61K 31/74; A61K 6/00; A61K 7/00

(52) U.S. Cl. 424/401; 424/78.03; 514/945; 514/947

(57) ABSTRACT

A cleansing composition comprising:

(A) glycerin;

(B) one or more emollients;

(C) a first surfactant comprising a salt of cocoyl isethionate;

(D) a second surfactant where said second surfactant comprises one or more members of the group consisting on an anionic surfactant, an alkyl phosphate, a salt of an alkyl phosphate, and an amphoteric surfactant; and

(E) water.

### FIELD OF THE INVENTION

This invention relates to anhydrous cream cleanser formulations which exhibit good aesthetics, spreadability, and foaming. These cleansers also demonstrate stability on aging.

### BACKGROUND OF THE INVENTION

A mild depositing cleanser is disclosed in Ramirez et al., U.S. Pat. No. 5,409,706. This cleanser includes an essentially anhydrous oil and surfactant mixture dispersed in a continuous glycerin phase. This formulation exhibits poor in-use aesthetics such as, for example, poor spreadability of the cream when contacted with water which leads to poor in-use consumer performance attributes. Another deficiency is low amounts of foam. This cleanser also exhibits poor stability in that the cream often displays syneresis of both oils (lipophilic at the top of the cream) and glycerin (at the bottom). Even when left at room temperature for relatively short periods of time, tube samples of the cream ooze clear liquids. High levels of Carbopol are necessary to maintaining even short term stability.

The present inventors have discovered a cleanser formulation which overcomes these deficiencies.

### SUMMARY OF THE INVENTION

The invention includes a cleansing composition comprising:

(A) glycerin;

(B) one or more emollients;

(C) a first surfactant comprising a salt of cocoyl isethionate;

(D) a second surfactant where said second surfactant comprises one or more members of the group consisting on an anionic surfactant, an alkyl phosphate, a salt of an alkyl phosphate, and an amphoteric surfactant; and

(E) water.

The composition may further comprise:

(F) a polymeric thickener.

### DETAILED DESCRIPTION OF THE INVENTION

The present inventors have discovered cleansing compositions with improved foaming, stability and aesthetics. These compositions comprise glycerin; one or more emollients, a first surfactant; a second a second surfactant where said second surfactant comprises one or more members of the group consisting on an anionic surfactant, an alkyl phosphate, a salt of an alkyl phosphate, and an amphoteric surfactant; and water Glycerin is used in the compositions of the invention and is present in percent by weight, based upon 100 percent by weight of total composition. Typically glycerin is present in an amount ranging from about 30 to about 60 percent by weight, preferably, from about 35 to about 50 percent by weight, and most preferably from about 45 to about 46 percent by weight.

Emollients suitable for use in the compositions of the present invention include but are not limited to petrolatum; mineral oils; soybean oil; and esters such as, for example, isopropyl myristate, isopropyl palmitate, 1-decene polymer (hydrogenated), C₁₂-C₁₅ alkyl benzoate (also known as C₁₂₋₁₅ alcohol benzoate under the tradename Finisolv TN), and C₁₂-C₁₅ alkyl benzoate esters. Typically, said one or more emollients comprises C₁₂-C₁₅ alkyl benzoate and at least one other emollient selected from the group consisting of petrolatum, mineral oils, and soybean oil. The preferred said one or more emollients are a mixture of C₁₂-C₁₅ alkyl benzoate and petrolatum. The particularly preferred said one or more emollients comprises C₁₂-C₁₅ alkyl benzoate in an amount from about 1 to about 12 percent by weight, more preferably, from about 2 percent to about 10 percent, based on the total weight of the composition, where the balance of the weight of said one or more emollients comprises at least one other emollient.

Typically said one or more emollients are present, based on the total weight of the composition, in an amount ranging from about 20 to about 45 percent by weight, preferably, from about 25 to about 35 percent by weight and most preferably about 32 percent by weight. In the case of oil and ester emollients, a small amount of microcrystalline wax may be added to produce a cosmetically acceptable cream. If used, microcrystalline wax will be present, based on the total weight of the composition, in an amount from about 4 to about 10 percent by weight.

The first surfactant comprises a salt of cocoyl isethionate which includes, but is not limited to, sodium or ammonium cocoyl isethionate. Typically salts of cocoyl isethionate are present, based on the total weight of the composition, in an amount ranging from about 3 to about 19 percent by weight, preferably, from about 5 to about 15 percent by weight, and most preferably about 7.2 percent by weight.

The second surfactant comprises one or more members of the group consisting of an anionic surfactant, an alkyl phosphate, a salt of an alkyl phosphate, and an amphoteric surfactant. Typically said second surfactant comprises an anionic surfactant and alkyl phosphate.

Suitable anionic surfactants include, but are not limited to, sodium lauryl sulfate, sodium salts of fatty acid taurate, acyl glutamates, -olefin sulfonates, and sarcosinates such as, for example, sodium lauroyl sarcosinates. The preferred anionic surfactants is sodium lauryl sulfate

Suitable alkyl phosphates include, but are not limited to, C₉-C₁₅ alkyl phosphates such as, for example, Arlatone MAP concentrate (ICI Surfactants-Wilmington, Del.), lauryl phosphate acid, known as Rhodafac DV4922 (Rhodia-N.J.). The alkyl phosphates may be neutralized by neutralizing agents which include but are not limited to organic or inorganic bases where the preferred neutralizing agent is triethanolamine. These components can be neutralized to a pH ranging from about 5 to about 8, preferably from about 6.5 to about 7.5, and most preferably from about 6.8 to about 7.2. Aside from the alkyl phosphates, other components of the composition by be neutralized. Any components described as neutralized in this composition re neutralized using the neutralizing agents described above where the neutralized pH is as mentioned above. The preferred alkyl phosphates are monoalkyl phosphates and salts thereof.

Suitable amphoteric surfactants are betaines, where the preferred betaine is cocamidopropyl betaine.

The amount of the secondary surfactant included in the composition is typically an amount effective to increase the softness of the composition. Preferably, the weight ratio of the secondary surfactant to cocoyl isethionate salt ( first surfactant) ranges from about 1:6 to about 1:1 and the combination is about 8 percent by weight, based upon 100 percent by weight of total composition. While the precise amount of secondary surfactant employed will depend upon the particular surfactant employed, normally it will be present in an amount ranging from about 1 to about 20 weight percent, based upon 100 percent by weight of total composition. For example, the amount of sodium lauryl sulfate when used as the secondary surfactant, will range from about 1 to about 5 percent by weight, based upon 100 percent by weight of total composition. Preferably, the weight ratio of sodium lauryl sulfate to sodium cocoyl isethionate in the composition ranges from about 1:3 to about 1:6.

Effective amounts, based upon 100 percent by weight of total composition, of other secondary surfactants are as follows:

| Secondary Surfactant | Effective Amount |
|---|---|
| Sodium salts of fatty acid taurate | About 1 to about 5 weight percent |
| Acyl glutamates | About 1 to about 19 weight percent |
| -olefin sulfonates | About 1 to about 15 weight percent |

Water is present in an amount, based on the total weight of the composition, of 5 percent by weight or greater. Preferably, the amount of water ranges from about 5 to about 20 percent by weight and more preferably from about 5 to about 15 percent by weight, based upon 100 percent by weight total composition. Most preferably, water is present in an amount, based on the total weight of the composition, of from about 5 percent to about 12 percent.

Other components may be added to the compositions of the invention. For example, suitable polymer thickeners may be added. Said thickeners include but are not limited to, gums such as xantham gum, an acrylate/C₁₀-C₃₀ cross polymer such is that sold under the trade name Carbomer by B. F. Goodrich-Cleveland-Ohio, and carboxypolymethylene polymers (Carbopol). Thickeners such as Carbomer can be neutralized using the neutralizing agents described above. Preferably, the thickeners are present in an amount, based on the total weight of the composition, from about 0.1 to about 0.7 percent, most preferably, about 0.3 percent.

In addition to the foregoing components, foam boosters may be incorporated into the compositions of the present invention. Suitable foam enhancers include potassium polymetaphosphate, n-pentane, isopentate, sodium lauryl sulfoacetate, amides, and sarcosinates. These materials will enhance the foam produced when the present compositions are exposed to water during use.

The compositions of this invention may also contain additives such as fragrances, colorants, sugar, and sugar derivatives, UV stabilizers, antioxidants, preservatives, sun screens, and the like to improve the texture, appearance, and user perception of the compositions. Additionally, active ingredients may be incorporated in the present compositions. Such active ingredients include, but are not limited to deodorants; medicaments such as, for example coal-tar, benzoyl peroxide, vitamin A and vitamin E and derivatives thereof such as, for example, retinol or tretinoin and antibacterial agents such as, for example, triclosan, PVP-iodine and salicylic acid.

The compositions of the present invention may be formulated using cosmetic and pharmaceutical vehicles. The compositions may be formulated in any vehicle which may be applied to human skin, where such vehicles include but are not limited to mists, gel, liquids, creams, aerosols, and oils. Typically the compositions are formulated as creams, liquids, or gels.

The compositions of the present invention are useful in delivering skin enhancing active agents such as for example, retinol. Delivery is typically by topical application to an animal, preferably a mammal, but most preferably a human, in need of the active agent. Typical amounts administered will be skin enhancing effective amounts of the composition containing the active agent.

Further, the inventors have discovered that the foaming, stability and aesthetics of cleansing compositions may be improved even further, by employing one or more of the following steps adding a controlled amount of polymeric thickener to the cleansing compositions, neutralizing the components of the cleansing compositions.

Typically, the aforementioned thickeners may be added to the cleansing compositions at the ranges discussed previously. The components of the cleansing compositions may be neutralized using the neutralizing agents and amounts discussed previously. The best cleansing compositions are prepared with additional thickeners and neutralized components.

Still further, the inventors have discovered that adding low levels of water to cleansing formulations with high glycerin and high emollient concentrations improves the foaming, stability and aesthetics of said cleansing formulations.

Aside from the cleansing compositions of the invention, the addition of low levels of water to cleansing formulations improves the foaming aesthetics, and stability of said cleansing formulations. Typically said cleansing formulations have glycerin present in an amount, based upon the total weight of the formulation, ranging from about 30 to about 60 percent by weight, preferably, from about 35 to about 50 percent by weight, and most preferably from about 45 to about 46 percent by weight; and emollients present in an amount, based upon the total weight of the formulation, ranging from about 20 to about 45 percent by weight, preferably, from about 25 to about 35 percent by weight and most preferably about 32 percent by weight. These cleansing compositions are improved by the addition of water in an amount, based on the weight of the total composition of 5 percent by weight or greater. Preferably, the amount of water ranges from about 5 to about 20 percent by weight and more preferably from about 5 to about 15 percent by weight, based upon 100 percent by weight total composition. Most preferably, water is present in an amount, based on the total weight of the composition, of from about 5 percent to about 12 percent.

Although the compositions of the present invention may be prepared by any method of vigorously mixing the ingredients together, preferably, these compositions are prepared by high shear blending. The high shear blending preferably is continued through cooling to temperature below the freezing range of the emollients to keep the oil phase dispersed throughout the freezing range. For example, in the case of petrolatum, this cooling would be below 45° C. The order of addition of components is not critical.

Typically the method of preparing the cleansing compositions of the invention comprises

(1) mixing glycerin and water together; and

(2) adding to the glycerin and water mixture emollients and surfactants.

All components and amounts of glycerin, water, emollients and surfactants are as previously described above.

In order to illustrate the invention the following example is included. These examples do not limit the invention. They are meant only to suggest a method of practicing the invention. Those knowledgeable in the preparation cleansing compositions as well as other specialties may find other methods of practicing the invention. However, those methods are deemed to be within the scope of this invention.

### EXAMPLES

### Example 1

Composition A was prepared from the ingredients listed in Table A. Three phases, a glycerin phase, an Oil Phase Part A and an Oil Phase Part B were prepared separately and mixed together to form the compositions.

Glycerin Phase

Add glycerin to a suitable beaker and agitate. Sift in acrylates/C10-30 alkyl cross polymer, methylparaben, propylparaben, and tetrasodium EDTA. Transfer to a homogenizer and begin to homogenize and heat to 80° C. Agitate until polymer slurry is uniform (no fish eyes evident). Add water and adjust temperature to 80° C. Neutralize with triethanolamine. Maintain at 80° C and continue to agitate on homogenizer.

Oil Phase Part A

Add petrolatum to a suitable sized beaker and begin to heat to 80° C. When petrolatum begins to soften and liquefy begin to homogenize (low setting) with a gifford wood homog. Add lauryl alkyl phosphate acid and neutralize with triethanolamine 99%. Homogenize. Add the sodium cocoyl isethinonate and allow to wet out in melted oils. Add sodium C₁₄₋₁₆ olefin sulfonate and allow to wet out in melted oils. Maintain agitation, homogenization and temperature at 80° C. Add potassium polyphosphate. Maintain homogenizer agitation and temperature at 80° C. Add titanium dioxide and mix until uniform. Maintain homogenizer agitation and temperature at 80° C.

Oil Phase Part B

In a separate beaker, heat the C₁₂₋₁₅ alkyl benzoate to 80° C. Add the BHT to the C12-15 alkyl benzoate, mix and allow to dissolve, then add the soybean oil to the mixture and allow to completely dissolve. Hold at 75-80° C. for phasing to the Esco Vessel.

Esco Vessel

Transfer the Oil Phase Part A components (80° C.) to the Esco vessel which is preset at 80 ° C. Start mixing with side scrapper agitation set at 20-40% of max. speed (Actual 35%). Draw a vacuum of -0.6 to -0.8 bar (Actual=−0.7 bar). Transfer the Glycerin phase components (80° C.) to a funnel hopper and add to the glycerin phase which continuing to mix. Mix with sweep for 5 minutes. Maintain a vacuum of −0.6 to −0.8 bar. Transfer the Oil Phase Part B components (80° C.) to the Esco vessel which is preset at 80° C. Start the homogenizer and homogenize for 10 minutes at 40% of maximum. If necessary adjust vacuum to −0.6 to −0.8 barand maintain heat at 80° C. for 10-15 minutes. Check the batch visually for a uniform appearance. Maintain vacuum, low to moderate 25-35 (actual=30) sweep speed, and low 20-25 (actual=20) homogenizer, and cool the batch to 45° C. When the batch is at 45° C. turn off the homogenizer and maintain sweep agitation. Add the fragrance and allow to mix for 20 minutes at 40-45 ° C. Cool to 35° C. with sweep agitation and fill and in 4×2 oz jars.

TABLE A

| CTFA Name | % Wt. |
|---|---|
| Glycerin | 45.350 |
| Acrylates/C₁₀₋₃₀ Alkyl Crosspolymer | 0.150 |
| Triethanolamine | 1.125 |
| Methylparaben | 0.200 |
| Propylparaben | 0.100 |
| Tetrasodium EDTA | 0.200 |
| Water | 10.000 |
| Petrolatum | 25.000 |
| Lauryl Alkyl Phosphate Acid | 0.625 |
| Sodium Cocoyl Isethionate | 7.200 |
| Sodium C₁₄₋₁₆ Olefin Sulfonate | 1.200 |
| Potassium Polyphosphate | 0.500 |
| Titanium Dioxide | 0.300 |
| C₁₂₋₁₅ Alkyl Benzoate | 2.500 |
| BHT | 0.100 |
| Soybean Oil | 5.250 |
| Fragrance | 0.200 |
| | 100.000 |

### Example 2

Composition B was prepared from the ingredients listed in Table B, by substantially the same method as Composition A.

TABLE B

| CTFA Name | % Wt. |
|---|---|
| Glycerin | 47.30 |
| Acrylates/C₁₀₋₃₀ Alkyl Crosspolymer | 0.15 |
| Triethanolamine | 0.50 |
| Water | 10.00 |
| Petrolatum | 25.00 |
| Disodium Cocamido MEA Sulfosuccinate | 0.960 |
| Cocamidopropyl Betaine | 0.240 |
| Sodium Cocoyl Isethionate | 7.20 |
| Potassium Polyphosphate | 0.500 |
| Titanium Dioxide | 0.300 |
| C₁₂₋₁₅ Alkyl Benzoate | 2.50 |
| BHT | 0.10 |
| Vitamin A Alcohol (and) Soybean Oil | 5.25 |
| | 100.00 |

### Example 3

Composition C was prepared from the ingredients listed in Table C, by substantially the same method as Composition A.

TABLE C

| CTFA Name | % Wt. |
|---|---|
| Glycerin | 48.250 |
| Acrylates/C₁₀₋₃₀ Alkyl Crosspolymer | 0.150 |
| Triethanolamine | 1.125 |
| Water | 10.000 |
| Petrolatum | 25.000 |
| C₉-C₁₅ Alkyl Phosphate Acid | 0.625 |
| Sodium Cocoyl Isethionate | 7.200 |
| Potassium Polyphosphate | 0.500 |
| Titanium Dioxide | 0.300 |
| C₁₂₋₁₅ Alkyl Benzoate | 6.7 50 |
| BHT | 0.100 |
| | 100.000 |

### Example 4

Composition D was prepared from the ingredients listed in Table D, by substantially the same method as Composition A.

TABLE D

| CTFA Name | % Wt. |
|---|---|
| Glycerin | 47.800 |
| Acrylates/C₁₀₋₃₀ Alkyl Crosspolymer | 0.150 |
| Triethanolamine | 0.525 |
| Water | 10.000 |
| Petrolatum | 25.000 |
| C₁₂₋₁₅ Alkyl Benzoate | 7.000 |
| C₉₋₁₅ Alkyl Phosphate Acid | 0.625 |
| Sodium Cocoyl Isethionate | 6.800 |
| Sodium C₁₄₋₁₆ Olefin Sulfonate | 1.200 |
| Potassium Polyphosphate | 0.500 |
| Titanium Dioxide | 0.300 |
| BHT | 0.100 |
| TOTAL | 100.000 |

### Example 5

Composition E was prepared from the ingredients listed in Table E, by substantially the same method as Composition A.

TABLE E

| CTFA Name | % Wt. |
|---|---|
| Glycerin | 42.80 |
| Deionized Water | 15.00 |
| Acrylates/C₁₀₋₃₀ Alkyl Crosspolymer | 0.15 |
| Triethanolamine | 0.15 |
| Petrolatum | 25.00 |
| C₁₂₋₁₅ Alkyl Benzoate | 7.00 |
| C₉₋₁₅ Alkyl Phosphate Acid | 0.63 |
| Triethanolamme | 0.38 |
| BHT | 0.10 |
| Sodium Cocoyl Isethionate | 6.80 |
| Sodium C₁₄₋₁₆ Olefin Sulfonate | 1.20 |
| Potassium Polyphoaphate | 0.60 |
| Titanium Dioxide | 0.30 |

### Example 6

Composition F was prepared from the ingredients listed in Table F, by substantially the same method as Composition A.

TABLE F

| CTFA Name | % Wt. |
|---|---|
| Glycerin | 32 |
| Distilled Water | 10 |
| Acrylates/C₁₀₋₃₀ Alkyl Crosspolymer | 0.5 |
| Tnethanolamine | 0.36 |
| Polyacrylamide (and) Isoparafin (and) Laureth 7 | 1 |
| Petrolatum | 21.4 |
| C₉₋C₁₅ Alkyl Phosphate Acid | 0.6 |
| Sodium Cocoyl Isethionate | 5 |
| Sodium C₁₄₋₁₆ Olefin Sulfonate | 2 |
| Potassium Polymetaphosphate | 0.6 |
| Titanium Dioxide | 0.3 |
| Zinc Lactate | 2 |
| Triethanolamine | 0.74 |
| C₁₂₋₁₅ Alkyl Benzoate | 6.3 |
| Benzoyl Peroxide | 15.8 |
| C1215 Alyki Benzoate | 0.7 |
| Fragrance | 0.4 |
| Menthol | 0.4 |

### Example 7

Composition G, an anhydrous composition was prepared from the ingredients listed in Table G, by substantially the same method as Composition A. This anhydrous composition does not have any water of the Glycerin Phase.

TABLE G

| CTFA Name | % Wt. |
|---|---|
| Glycerin | 58.25 |
| Acrylates/C₁₀₋₃₀ Alkyl Crosspolymer | 0.15 |
| Triethanolamine | 025 |
| Petrolatum | 25.00 |
| C₉₋₁₅ Alkyl Phosphate | 0.625 |
| Triethanolamine | 0.375 |
| Sodium Cocoyl Isethionate | 7.20 |
| Potassium Polyphosphate | 0.500 |
| Titanium Dioxide | 0.300 |
| C₁₂₋₁₅ Alkyl Benzoate | 8.75 |
| BHT | 0.10 |

### Example 8

The improved spreadability of the cleansing compositions of the invention, composition E and F demonstrated by comparing the viscosity of those compositions with the viscosity of the anhydrous formulation, Composition G. The compositions were prepared and the initial viscosity was measured on a Brookfield DVI TF machine at 1.5 rpm. The results of the experiment are listed in Table H. The compositions were compared empirically as well. Compositions E and F have good spreadibility, increased flash foam, and generally better foaming properties than Composition G.

TABLE H

| Composition | Viscosity |
|---|---|
| E | 6,666 cPs |
| F | 40,000 cPs |
| G | 166,000 cPs |

What is claimed is:

1. A cleansing composition comprising:
(A) glycerin;
(B) one or more emollients;
(C) a first surfactant comprising a salt of cocoyl isethionate;
(D) a second surfactant where said second surfactant comprises one or more members of the group consisting on an anionic surfactant, an alkyl phosphate, a salt of an alkyl phosphate, and an amphoteric surfactant; and
(E) water.

2. The cleansing composition of claim 1 which further comprises
(F) a polymeric thickener.

3. The cleansing composition of claim 1 wherein said one or more emollients are selected from the group consisting of petrolatum, mineral oils, soybean oil, isopropyl myristate, isopropyl palmitate, 1-decene polymer (hydrogenated), C₁₂-C₁₅ alkyl benzoate, and C₁₂-C₁₅ alkyl benzoates esters.

4. The cleansing composition of claim 1 wherein said salt of cocoyl isethionated is selected from the group consisting of ammonium cocoyl isethionate and sodium cocoyl isethionate.

5. The cleansing composition of claim 1 wherein said second surfactant further comprises an anionic surfactant and an alkyl phosphate.

6. The cleansing composition of claim 5 wherein
said anionic surfactant is selected from the group consisting of sodium lauryl sulfate, sodium salts of fatty acid taurate, acyl glutamates, -olefin sulfonates, and sarcosinates; and
said alkyl phosphate is selected from the group consisting of C₉-C₁₅ alkyl phosphates and lauryl phosphate acid.

7. The cleansing composition of claim 6 wherein said alkyl phosphate is neutralized to a pH ranging from about 5 to about 8.

8. The cleansing composition of claim 2 wherein
glycerin is present in an amount of about 45 to about 46 percent by weight;
said one or more emollients are present in an amount of 32 percent by weight;
said first surfactant is present in an amount of about 7.2 percent by weight;
said second surfactant is present in an amount of about 1.2 percent to about 3.2;
water is present in an amount of about 5 percent to 12 percent; and said polymeric thickener is present in an amount of about 0.1 percent to about 0.7 percent.

9. The cleansing composition of claim 2 which further comprises a foam booster, skin enhancing active agents, and active ingredients.

10. The cleansing composition of claim 8 wherein said secondary surfactant comprises an anionic surfactant and an alkyl phosphate.

11. The cleansing composition of claim 10 wherein said alkyl phosphate and said polymeric thickener are neutralized.

12. The cleansing composition of claim 10 wherein
said one or more emollients comprise C₁₂-C₁₅ alkyl benzoate and petrolatum;
said first surfactant is sodium cocyl isethionate;
said second surfactant is sodium lauryl sulfate and an alkyl phosphate; and
said polymeric thickener is acrylate/C₁₀-C₃₀ cross polymer.

13. A method of preparing the cleansing compositions which comprises
(1) mixing glycerin and water together; and
(2) adding to the glycerin and water mixture emollients and surfactants.

14. The method of claim 13 wherein the emollients and surfactants are mixed together before they are added to the glycerin and water mixture.

15. The method of claim 13 where all components are mixed by high shear blending.
